(19) United States
(12) Patent Application Publication
(10) Pub. No.: US 2012/0142918 A1
(43) Pub. Date: Jun. 7, 2012

(54) LIGANDS

(76) Inventors: Ranbir Singh Padda, West Midlands (GB); Martin Barry Smith, Leicestershire (GB); Gordon Findlay Docherty, Birmingham (GB); Michael John Harrison, Staffordshire (GB)

(21) Appl. No.: 13/146,243

(22) PCT Filed: Jan. 26, 2010
(86) PCT No.: PCT/GB10/50112

§ 371 Date: Dec. 6, 2011

(30) Foreign Application Priority Data
Jan. 26, 2009 (GB) 0901231.1

(51) Int. Cl. C07F 9/6584; C07C 45/50; C07C 209/60; C07C 4/06; C07D 301/03; C07C 29/00; C07C 2/42; C07F 19/00; C07F 7/08

(52) U.S. Cl. 544/181; 544/214; 568/420; 564/1; 556/479; 549/523; 568/700; 585/350; 585/752

(57) ABSTRACT

A caged phosphine product is provided which can act as a ligand to form a metal complex. The metal complex can be used as a catalyst. The caged phosphine is a compound of formula (I):

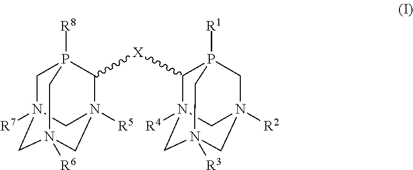

or a salt thereof;
wherein the groups R¹ and R⁸ each independently represent: (a) no substituent group; (b) an oxide substituent group ═O; (c) a sulphide substituent group ═S; (d) a selenide substituent group ═Se; (e) a C1-C8 alkyl substituent group; (f) a C6-C8 aryl substituent group or a 5 to 8 membered ring hetero aryl substituent group; or (g) a Lewis acid substituent group;
wherein the groups R², R³, R⁴, R⁵, R⁶ and R⁷ each independently represent: (1) no substituent group; (2) a hydrogen substituent group; (3) a C1-C8 alkyl substituent group; (4) a C1-C8 alkoxy substituent group; or (5) a C1-C8 acyl substituent group;
and wherein X represents a linking group that is selected from: (i) a C1-C12 alkylene linking group; (ii) an ether linking group; (iii) a C2-C6 alkenylene linking group; (iv) an ester linking group; (v) a (hetero)arylene linker; (vi) an amine linker; or (vii) a thioether linker.

The present invention relates to new phosphorus based compounds that can be used as ligands.

The compound 1,3,5-triaza-7-phosphaadarnantane (PTA) is a caged phosphine product that has been known since 1974, having been described in Heterocyclic Chemistry 11 407 (1974). PTA has the following structure:

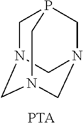

PTA is soluble in water and air stable. It can form metal complexes with various metals, including Rh, Ru, Pd, Ir, Cr, Mo, W, Au, Hg and Fe. PTA complexes may be used in aqueous phase catalysis or biphasic homogeneous catalysis. PTA may also be used as a catalyst in non complexed form, i.e. as an organocatalyst.

The invention provides, in a first aspect, a caged phosphine product which is a compound of formula (I):

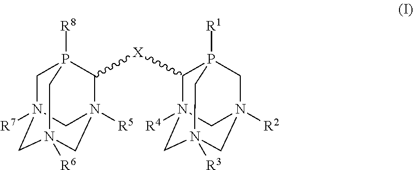

or a salt thereof;
wherein the groups R¹ and R⁸ each independently represent:
(a) no substituent group;
(b) an oxide substituent group ═O;
(c) a sulphide substituent group ═S;
(d) a selenide substituent group ═Se;
(e) a C1-C8 alkyl substituent group, such as methyl, ethyl or propyl;
(f) a C6-C8 aryl substituent group, such as phenyl, or a hetero aryl substituent group derived from a C5-C8 aryl substituent group, such as furyl, thiophenyl or pyridyl; or
(g) a Lewis acid substituent group, such as BH₃, BF₃, AlH₃, AlF₃, SiF₄ or SF₄;
wherein the groups R², R³, R⁴, R⁵, R⁶ and R⁷ each independently represent:
(1) no substituent group;
   (2) a hydrogen substituent group;
(3) a C1-C8 alkyl substituent group, such as methyl, ethyl or propyl;
(4) a C1-C8 alkoxy substituent group, such as methoxy or ethoxy; or
(5) a C1-C8 acyl substituent group, such as formyl or acetyl;
and wherein X represents a linking group that is selected from:
(i) a C1-C12 alkylene linking group, e.g. a C1-C8 alkylene linking group, such as methylene or ethylene or propylene or butylene or pentylene;
(ii) an ether linking group, such as —(CH₂)ₙO(CH₂)ₒ—, where n and o independently represent an integer of from 0 to 3, e.g. from 1 to 3;
(iii) a C2-C6 alkenylene linking group, such as ethenylene;
(iv) an ester linking group, such as —(CH₂)ₙCOO(CH₂)ₒ—, where n and o independently represent an integer of from 0 to 3, e.g. from 1 to 3;
(v) a (hetero)arylene linker, such as —(CH₂)ₙ(Ar)(CH₂)ₒ—, where n and o independently represent an integer of from 0 to 3, e.g. from 1 to 3, and Ar is a C6-C8 arylene substituent group, such as phenylene, or a 5 to 8 membered ring hetero arylene substituent group, such as furylene, thiophenylene or pyridylene;
(vi) an amine linker of formula —RxN(Rz)Ry—, for example wherein Rx and Ry are independently C1-C4 alkylene and Rz is H or C1-C4 alkyl, such as —CH₂N(CH₃)CH₂—; or
(vii) a thioether linker, such as —(CH₂)ₙS(CH₂)ₒ—, where n and o independently represent an integer of from 0 to 3, e.g. from 1 to 3.

In the present application, reference to a 5 to 8 membered ring hetero arylene substituent group means a hetero arylene group derived from a C5-C8 arylene substituent group, in other words a C5-C8 arylene substituent group with one or more of the carbon atoms replaced by a hetero atom. Examples include furylene, thiophenylene and pyridylene.

Equally, reference to a 6 membered ring hetero arylene substituent group means a hetero arylene group derived from a C6 arylene substituent group, in other words a C6 arylene substituent group with one or more of the carbon atoms replaced by a hetero atom.

It will be understood that when any of the groups R², R³, R⁴, R⁵, R⁶ and R⁷ represent no substituent group the amine is tertiary whereas when any of these groups represent H, alkyl, alkoxy or acyl there is a quaternary ammonium.

In one embodiment, the following applies:
the groups R¹ and R⁸ each independently represent:
(a) no substituent group;
(b) an oxide substituent group ═O;
(c) a sulphide substituent group ═S;
(d) a selenide substituent group ═Se;
(e) a C1-C8 alkyl substituent group, such as methyl, ethyl or propyl;
(f) a C6-C8 aryl substituent group, such as phenyl, or a hetero aryl substituent group derived from a C5-C8 aryl substituent group, such as furyl, thiophenyl or pyridyl; or
(g) a Lewis acid substituent group, such as BH₃, BF₃, AlH₃, AlF₃, SiF₄ or SF₄;
   and
the groups R², R³, R⁴, R⁵, R⁶ and R⁷ each independently represent:
(1) no substituent group;
(2) a hydrogen substituent group;
(3) a C1-C8 alkyl substituent group, such as methyl, ethyl or propyl;
(4) a C1-C8 alkoxy substituent group, such as methoxy or ethoxy; or
(5) a C1-C8 acyl substituent group, such as formyl or acetyl;
   and
X represents a linking group that is selected from:
(i) a C1-C6 alkylene linking group, such as methylene or ethylene;
(ii) an ether linking group, such as —(CH₂)ₙO(CH₂)ₒ—, where n and o independently represent an integer of from 0 to 3, e.g. from 1 to 3;
(iii) a C2-C6 alkenylene linking group, such as ethenylene;
(iv) an ester linking group, such as —(CH₂)ₙCOO(CH₂)ₒ—, where n and o independently represent an integer of from 0 to 3, e.g. from 1 to 3;
(v) a (hetero)arylene linker, such as —(CH₂)ₙ(Ar)(CH₂)ₒ—, where n and o independently represent an integer of from 0 to 3, e.g. from 1 to 3, and Ar is a C6-C8 arylene substituent group, such as phenylene, or a 5 to 8 membered ring hetero arylene substituent group, such as furylene, thiophenylene or pyridylene;
(vi) an amine linker of formula —RxN(Rz)Ry—, for example wherein Rx and Ry are independently C1-C4 alkylene and Rz is H or C1-C4 alkyl, such as —CH₂N(CH₃)CH₂—; or
(vii) a thioether linker, such as —(CH₂)ₙS(CH₂)ₒ—, where n and o independently represent an integer of from 0 to 3, e.g. from 1 to 3.

Preferably, R¹ and R⁸ are the same.

In one embodiment, the groups R¹ and R⁸ each independently represent:
(a) no substituent group;
(b) an oxide substituent group ═O;
(c) a sulphide substituent group ═S;
(d) a selenide substituent group ═Se;
(e) a C1-C6 alkyl substituent group, such as methyl, ethyl or propyl;
(f) a C6-C8 aryl substituent group, such as phenyl, or a hetero aryl substituent group derived from a C5-C8 aryl substituent group, such as furyl, thiophenyl or pyridyl; or
(g) a Lewis acid substituent group, such as BH₃, BF₃, AlF₃, SiF₄ or SF₄.

Preferably, the groups R¹ and R⁸ each independently represent:
(a) no substituent group;
(b) an oxide substituent group ═O;
(c) a sulphide substituent group ═S;
(d) a selenide substituent group ═Se;
(e) a C1-C4 alkyl substituent group, such as methyl, ethyl or propyl;
(f) a C6 aryl substituent group, or a 6 membered ring hetero aryl substituent group, such as pyridyl; or
(g) a Lewis acid substituent group, such as BH₃, BF₃, AlH₃, AlF₃, SiF₄ or SF₄.

In one embodiment, the groups R¹ and R⁸ each independently represent:
(a) no substituent group;
(b) an oxide substituent group ═O;
(c) a sulphide substituent group ═S; or
(d) a selenide substituent group ═Se.

In one embodiment, the groups R¹ and R⁸ are the same and each represents:
(a) no substituent group;
(b) an oxide substituent group ═O;
(c) a sulphide substituent group ═S; or
(d) a selenide substituent group ═Se.

In one embodiment, the groups R¹ and R⁸ each represent:
(a) no substituent group.

Preferably, R² and R⁷ are the same.

Preferably, R³ and R⁶ are the same.

Preferably, R⁴ and R⁵ are the same.

In one embodiment, R², R³, R⁴, R⁵, R⁶ and R⁷ are all the same.

It may be that at least one of R², R³, R⁴, R⁵, R⁶ and R⁷ represents no substituent group. For example, two, three, four, five, or all six, of R², R³, R⁴, R⁵, R⁶ and R⁷ may represent no substituent group.

In one embodiment, only one, or only two, of R², R³, R⁴, R⁵, R⁶ and R⁷ represents a substituent group selected from options (2), (3), (4) and (5).

In another embodiment, three or more of R², R³, R⁴, R⁵, R⁶ and R⁷ represents a substituent group selected from options (2), (3), (4) and (5).

It may be, for example, that four or five of R², R³, R⁴, R⁵, R⁶ and R⁷ may represent no substituent group, whilst the remainder are selected from hydrogen substituent groups or C1-C8 alkyl substituent groups; preferably the remainder are selected from C1-C6 alkyl substituent groups; most preferably the remainder are selected from C1-C4 alkyl substituent groups, such as methyl or ethyl groups.

In a preferred embodiment the groups R², R³, R⁴, R⁵, R⁶ and R⁷ each independently represent:
(1) no substituent group;
(2) a hydrogen substituent group;
(3) a C1-C6 alkyl substituent group such as methyl, ethyl or propyl;
(4) a C1-C6 alkoxy substituent group such as methoxy or ethoxy;
(5) a C1-C6 acyl substituent group such as formyl or acetyl.

In a further preferred embodiment the groups R², R³, R⁴, R⁵, R⁶ and R⁷ each independently represent:
(1) no substituent group;
(2) a hydrogen substituent group;
(3) a C1-C4 alkyl substituent group such as methyl, ethyl or propyl;
(4) a C1-C4 alkoxy substituent group such as methoxy or ethoxy;
(5) a C1-C4 acyl substituent group such as formyl or acetyl.

In one preferred embodiment, the groups R², R³, R⁴, R⁵, R⁶ and R⁷ each independently represent: no substituent group or a C1-C4 alkyl substituent group, such as methyl, ethyl or propyl.

In one embodiment, X represents a linking group that is selected from:
(i) a C1-C12 alkylene linking group, e.g. a C1-C8 alkylene linking group, for example a C1-C6 alkylene linking group, such as methylene, ethylene, propylene, butylene or pentylene;
(ii) an ether linking group, such as —(CH₂)ₙO(CH₂)ₒ—, where n and o independently represent an integer of from 1 to 3, and n+o equals 4 or less;
(iii) a C2-C4 alkenylene linking group such as ethenylene;
(iv) an ester linking group —(CH₂)ₙCOO(CH₂)ₒ—, where n and o independently represent an integer of from 1 to 3, and n+o equals 4 or less;
(v) a (hetero)arylene linker, such as —(CH₂)ₙ(Ar)(CH₂)ₒ—, where n and o independently represent an integer of from 1 to 3, and n+o equals 4 or less, and Ar is a C6-C8 arylene substituent group, such as phenylene, or a 5 to 8 membered ring hetero arylene substituent group, such as furylene, thiophenylene or pyridylene;
(vi) an amine linker of formula —RxN(Rz)Ry—, for example wherein Rx and Ry are C1-C4 alkylene, e.g. C1 or C2 alkylene, and Rz is H or C1-C4 alkyl, e.g. C1 or C2 alkyl; or
(vii) a thioether linker such as —(CH₂)ₙS(CH₂)ₒ—, where n and o independently represent an integer of from 1 to 3, and n+o equals 4 or less.

In one embodiment, X represents a linking group that is selected from:
(i) a C1-C6 alkylene linking group, e.g. a C1-C5 alkylene linking group, such as methylene or ethylene;
(ii) an ether linking group, such as —(CH₂)ₙO(CH₂)ₒ—, where n and o independently represent an integer of from 1 to 3, and n+o equals 4 or less;
(iii) a C2-C4 alkenylene linking group such as ethenylene;
(iv) an ester linking group —(CH₂)ₙCOO(CH₂)ₒ—, where n and o independently represent an integer of from 1 to 3, and n+o equals 4 or less;
(v) a (hetero)arylene linker, such as —(CH₂)ₙ(Ar)(CH₂)ₒ—, where n and o independently represent an integer of from 1 to 3, and n+o equals 4 or less, and Ar is a C6-C8 arylene substituent group, such as phenylene, or a 5 to 8 membered ring hetero arylene substituent group, such as furylene, thiophenylene or pyridylene;
(vi) an amine linker of formula —RxN(Rz)Ry—, for example wherein Rx and Ry are C1-C4 alkylene, e.g. C1 or C2 alkylene, and Rz is H or C1-C4 alkyl, e.g. C1 or C2 alkyl; or
(vii) a thioether linker such as —(CH₂)ₙS(CH₂)ₒ—, where n and o independently represent an integer of from 1 to 3, and n+o equals 4 or less.

In one embodiment, X represents a linking group that is selected from:
(i) a C1-C4 alkylene linking group such as methylene or ethylene;
(ii) an ether linking group, such as —(CH₂)ₙO(CH₂)ₒ—, where n and o independently represent an integer of from 1 to 3, and n+o equals 4 or less;
(iii) a C2-C4 alkenylene linking group such as ethenylene;
(iv) an ester linking group —(CH₂)ₙCOO(CH₂)ₒ—, where n and o independently represent an integer of from 1 to 3, and n+o equals 4 or less;
(v) a (hetero)arylene linker, such as —(CH₂)ₙ(Ar)(CH₂)ₒ—, where n and o independently represent an integer of from 1 to 3, and n+o equals 4 or less, and Ar is a C6-C8 arylene substituent group, such as phenylene, or a 5 to 8 membered ring hetero arylene substituent group, such as furylene, thiophenylene or pyridylene;
(vi) an amine linker of formula —RxN(Rz)Ry—, for example wherein Rx and Ry are C1-C4 alkylene, e.g. C1 or C2 alkylene, and Rz is H or C1-C4 alkyl, e.g. C1 or C2 alkyl; or
(vii) a thioether linker such as —(CH₂)ₙS(CH₂)ₒ—, where n and o independently represent an integer of from 1 to 3, and n+o equals 4 or less.

It is preferred that X represents a linking group that is a C1-C12 alkylene linking group, more preferably a C1-C8 alkylene linking group. In one embodiment, the alkylene linking groups are straight chain. In another embodiment, the linking groups are branched alkylene groups. For example, X may represent a linking group that is a C1-C12 straight chain alkylene linking group (such as a C1-C8 or C1-C6 straight chain alkylene linking group) or a C2-C12 branched chain alkylene linking group (such as a C2-C8, or C2-C6, or C3-C6 branched chain alkylene linking group). Preferably, X represents a linking group that is a C1-C6 alkylene linking group, more preferably a C1-C5 alkylene linking group. It may therefore be methylene, ethylene, propylene, butylene or pentylene. In one embodiment, X represents a linking group that is a C1-C4 alkylene linking group, such as methylene, ethylene or propylene.

In one preferred embodiment, the groups R², R³, R⁴, R⁵, R⁶ and R⁷ each independently represent: no substituent group or a C1-C4 alkyl substituent group, such as methyl, ethyl or propyl, and X represents a linking group that is a C1-C12 alkylene linking group, such as methylene, ethylene, propylene, butylene or pentylene.

In one preferred embodiment, the groups R², R³, R⁴, R⁵, R⁶ and R⁷ each independently represent: no substituent group or a C1-C4 alkyl substituent group, such as methyl, ethyl or propyl, and X represents a linking group that is a C1-C8 alkylene linking group, such as methylene, ethylene, propylene, butylene or pentylene.

In one preferred embodiment, the groups R³, R⁴, R⁵, R⁶ and R⁷ each independently represent: no substituent group or a C1-C4 alkyl substituent group, such as methyl, ethyl or propyl, and X represents a linking group that is a C1-C6 alkylene linking group, such as methylene, ethylene, propylene, butylene or pentylene.

In the present specification, where reference is made to alkyl or alkylene groups these may be branched or, preferably, straight chain. An alkyl group may be a cycloalkyl group if it has 6 carbon atoms or more.

When reference is made to any hydrocarbon groups, in particular alkyl groups, aryl groups and acyl groups, they may be substituted or, preferably, unsubstituted. When substituted, one or more of the hydrogens of the hydrocarbon may be replaced with a substituent group such as fluoro, chloro, hydroxyl, C1-4 alkoxy, or NR₂ where each R is independently hydrogen or C1-4 alkyl.

In one embodiment, the product is of formula (II):

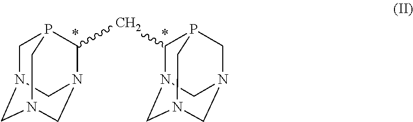

In other words, R¹, R², R³, R⁴, R⁵, R⁶, R⁷ and R⁸ each represent no substituent group, and X is methylene.

In another embodiment, the product is of formula (III):

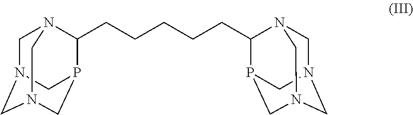

In other words, R¹, R², R³, R⁴, R⁵, R⁶, R⁷ and R⁸ each represent no substituent group, and X is pentylene.

When the caged phosphine product of the invention is a salt of the compound of formula (I), the counter-ion may be any suitable anion, for example it may be selected from iodine, bromine, chlorine, fluorine, sulphate, sulphite, phosphate, nitrate, and nitrite.

The compounds of formula (I) have two centres of chirality, at each of the two carbon centres adjacent the linking group X.

The compounds therefore exist in the form of a number of chiral enantiomers and all such chiral enantiomers are covered by the present invention. Specifically, the (R,R), (R,S), (S,R) and (S,S) enantiomers are all envisaged by the present invention.

In one embodiment, the product of the first aspect may be provided as a single chiral enantiomer. In another embodiment, the product of the first aspect may be provided as a mixture of two or more chiral enantiomers.

The stereogenic carbons located close to each phosphorus are believed to be advantageous, because when metal complexes are formed the metal will coordinate at the phosphorus.

Accordingly, the present invention provides an advantageous caged phosphine product in that the product is a chiral chelating phosphine, with centres of chirality close to the coordination centres.

When R² and R³ are not the same then the cage is also chiral and each nitrogen is chiral. Equally, when R⁶ and R⁷ are not the same then the cage is chiral and each nitrogen is chiral. Either of these embodiments may therefore advantageously be selected.

The caged phosphine product may be prepared by a method that involves:
(A) providing 1,3,5-triaza-7-phosphaadamantane, or a substituted derivative thereof having the required substituent groups to provide R¹ to R⁸.
(B) deprotonating this phosphaadamantane;
(C) adding a source of the linking group X.

PTA products with substitution at the phosphorus are known and available to the skilled man. See, for example, Phosphorus, Sulfur, and Silicon and the Related Elements, Volume 48, Issue 1-4 Mar. 1990, pages 37-40 which describes, inter alia, PTA oxide, sulphide and selenide.

PTA products with substitution at the nitrogen are also known and available to the skilled man. For example, Journal of Molecular Structure: THEOCHEM, Volume 894, Issues 1-3, 30 Jan. 2009, pages 59-63 refers to N-methyl PTA.

Additionally, Coordination Chemistry Reviews, Volume 248, Issues 11-12, June 2004, pages 955-993 describes various P-substituted and N-substituted PTA products, including N-protonated PTA; N-alkylated PTA; P-alkylated PTA; 1,3,5-triaza-7-phosphaadamantane oxide; 1,3,5-triaza-7-phosphaadamantane sulfide and 1,3,5-triaza-7-phosphaadamantane selenide.

The skilled man may therefore readily obtain a suitable starting material, depending upon his required R¹ to R⁸ groups, using known techniques and products.

The deprotonation in step (B) may be carried out using a base, e.g. alkyl lithium, in particular a C1-C4 alkyl lithium such as methyl lithium, n-butyl lithium or t-butyl lithium.

The deprotonation in step (B) may be carried out in an inert organic solvent. Suitable inert organic solvents include ethers, such as diethyl ether, tetrahydrofuran, dioxane and glycol ethers, and C5-C8 hydrocarbons, such as pentanes, hexanes, cyclohexane and iso-octane.

Step (B) may be carried out at room temperature, but temperatures of from −78° C. to +130° C. may be considered.

Step (B) is suitably carried out in an inert atmosphere, e.g. a nitrogen atmosphere. Alternatively, it may be carried out in air.

Preferably, the source of X in step (C) is provided as XY₂, where Y is a halogen, e.g. Br or C1. For example a C1-12 alkylene or C1-12 alkenylene, such as a C1-6 alkylene or C1-6 alkenylene (e.g. a C1-4 alkylene or C1-4 alkenylene) may be provided in the form of a dihalide, such as a dibromide. A (hetero)arylene —(CH₂)ₙ(Ar)(CH₂)ₒ— or an amine —RxN(Rz)Ry— may also be provided in the form of a dihalide. The ester, thio ester or ether linking groups of the invention can also be provided as dihalides.

Examples of the source of X include, but are not limited to: dibromomethane, dibromoethane (e.g. gem-dibromoethane), dibromopropane (e.g. 1,2-dibromopropane), dibromobutane (e.g. 2,3-dibromobutane) and dibromopentane (e.g. 1,5-dibromopentane).

Step (C) may be carried out in an inert organic solvent, such as those mentioned above, and may be carried out at room temperature.

The invention also provides, in a second aspect, a metal complex comprising a metal M coordinated with a ligand which is a compound of formula (I) as defined above.

The metal complex may be a complex of a single metal atom or ion with one or more ligands. Alternatively, the metal complex may be a complex of a two or more metal atoms or ions with one or more ligands. In this case, the metal atoms or ions are preferably the same.

There may be one or two or more ligands in the complex.

Only one of the ligands need be a compound of formula (I). In particular the metal M may be coordinated to further ligands which need not be a compound of formula (I).

In one embodiment, the complex may include one or more functional groups as well as the one or more ligands. These functional groups may, for example, be selected from carbonyl groups, alkyl groups (e.g. C1-12 straight or branched alkyl, such as C1-8 straight or branched alkyl, e.g. C1-4 straight or branched alkyl), halide groups (such as F, Cl, or Br), hydride groups, borohydride groups and hydrocarbon ring groups (e.g. aromatic ring groups, including cyclopentadienyl). There may, for example, be two, three, four, five or more of these groups. When there are two or more of these groups, they may be the same or different.

The metal M may be any metal atom or ion, for example it may be a transition metal (d block metal) or a lanthanide or actinide (f block metal). It may also be an alkali or alkaline earth metal (s block metal) or a p block metal (e.g. Group 13 or 14 metal).

In a preferred embodiment, the metal M is a transition metal (d block) atom or ion. It may be a Group 8, 9 or 10 transition metal, e.g. Fe, Ru, Co, Rh, Ir, Ni, Pd, Pt.

Other transition metals that can be used include Group 6 metals, such as Cr, W and Mo, and Group 11 and 12 metals, such as Au, Cu, Hg and Zn.

The metal may bind to the or each ligand via one or more phosphorus atom in the cage structure.

Overall the complex may be neutral or may be positively or negatively charged.

Examples of suitable transition metals for use as the metal M in particular include rhodium, ruthenium, rhenium, iridium, cobalt, nickel, platinum and palladium. Rhodium, ruthenium and iridium may be preferred among the above mentioned metals.

Specific examples of the said complexes of the present invention are described hereinbelow. However, these examples are not limiting.

The rhodium and iridium complexes can be represented by the following formulae:

[M L₂(P*P)]Y (IIa)

[M L₂ (P*P)]Y (IIb)

wherein in the said formulae: (P*P) represents the phosphine of formula (I), M represents rhodium or iridium, Y represents an anionic coordinating ligand, and L represents a neutral ligand.

The preferred rhodium or iridium complexes correspond to the formula (IIa) or (IIb) in which: L represents an olefin having from 2 to 12 carbon atoms and two L ligands can be joined to one another in order to form a linear or cyclic polyunsaturated hydrocarbon chain; L preferably representing 1,5-cyclooctadiene, norbornadiene or ethylene, Y represents an ionic coordinating ligand selected from: PF₆⁻, PCl₆⁻, BF₄⁻, BCl₄⁻, SbF₆⁻, SbCl₆⁻, BPh₄⁻, ClO₄⁻, CN⁻ and CF₃SO₃⁻ anions, halides (preferably Cl⁻ or Br⁻), a 1,3-diketonate, alkylcarboxylate or haloalkylcarboxylate anion with a C1-C4 alkyl group, and a phenylcarboxylate or phenoxide anion in which the benzene ring can be substituted by C1-C4 alkyl groups and/or halogen atoms.

Other iridium complexes can be represented by the formulae:

[IrL(P*P)]Y (IIIa)

[IrL(P*P)]Y (IIIb)

wherein in the said formulae (P*P), L and Y have the meanings given for the formulae (IIa) and (IIb).

As regards the ruthenium complexes, they preferentially correspond to the following formulae:

[RuY₁Y₂(P*P)] (IVa)

[RuY₁Y₂(P*P)] (IVb)

wherein in the said formulae: (P*P) represents the phosphine of formula (I), Y₁ and Y₂, are independently selected from a PF₆⁻, PCl₆⁻, BF₄⁻, BCl₄⁻, SbF_{b}⁻, SbCl₆⁻, BPh₄⁻, ClO₄⁻ or CF₃SO₃⁻ anion, a halide, more particularly chloride or bromide, or a carboxylate anion, preferentially acetate or trifluoroacetate.

Other ruthenium complexes capable of being used in the present invention correspond to the formulae hereinbelow:

[RuY₁Ar(P*P)]Y₂ (IVc)

[RuY₁Ar(P*P)]Y₂ (IVd)

in the said formulae: (P*P) represents the phosphine of formula (I), Ar represents benzene, p-methylisopropylbenzene or hexamethylbenzene, Y₁ represents a halide, preferably chloride or bromide, Y₂ represents an anion, preferably a PF₆⁻, PCl₆⁻, BF₄⁻, BCl₄⁻, SbCl₆⁻, BPh₄⁻, ClO₄⁻ or CF₃SO₃⁻ anion.

It is also possible to use complexes based on palladium and on platinum in the present invention.

Mention may be made, as more specific examples of the said complexes, of, inter alia, PdCl₂ (P*P) and PtCl₂ (P*P) in which (P*P) represents the phosphine of formula (I). In certain embodiments (P*P) may represent the phosphine of formula (II) or the phosphine of formula (III).

The complexes comprising the abovementioned diphosphine and the transition metal can be prepared according to the known processes described in the literature.

For the preparation of the ruthenium complexes, reference may in particular be made to the publication by J.-P. Genet [Acros Organics Acta, 1, No. 1, pp. 1-8 (1994)] and, for the other complexes, to the article by Schrock R. and Osborn J. A. [Journal of the American Chemical Society, 93, pp. 2397 (1971)].

They can be prepared in particular by reaction of the phosphine of formula (I) with the transition metal compound in a suitable organic solvent.

The reaction is carried out at a temperature of from ambient temperature (from 15 to 25′C) up to the reflux temperature of the reaction solvent.

Mention may be made, as examples of organic solvents, halogenated or non-halogenated aliphatic hydrocarbons and more particularly hexane, heptane, isooctane, decane, benzene; toluene, methylene chloride or chloroform; solvents of ether or ketone type and in particular diethyl ether, tetrahydrofuran, acetone or methyl ethyl ketone; or solvents of alcohol type, preferably methanol or ethanol.

The metal complexes according to the invention, recovered according to conventional techniques (filtration or crystallization), may be used as catalysts in organic reactions, as described below.

The present invention also provides, in a third aspect, the use of a complex in accordance with the second aspect as a catalyst.

The catalyst may, for example, be used to catalyse an organic reaction.

The organic reaction may, for example, be selected from hydrogenation (for example hydrogenation of carbon-carbon double bond or hydrogenation of carbon-heteroatom double bonds), hydroformylation, hydrosilylation, hydroamination, C—H bond activation (for example alkane C—H activation), C—C bond formation, cyclotrimerisation (for example cyclotrimerisation of alkenes), oxidation, epoxidation, dihydroxylation, and cycloadditions (e.g. [2+2+2] cycloadditions of diynes and isocyanates).

Preferably, the organic reaction is an asymmetric reaction.

The present invention will now be further described with reference to the following examples:

### EXAMPLE 1

### Production of PTA Methylene Dimer—Formula (II)

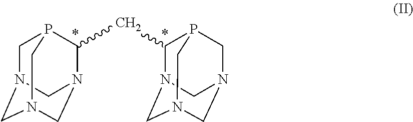

To prepare the product of formula (II) the following procedure may be followed: To a suspension of dried 1,3,5-triaza-7-phosphaadamantane (PTA) in THF is slowly added n-butyl lithium at −78° C. under a nitrogen atmosphere, in order to deprotonate the PTA. When the deprotonation reaction is observed to be completed, CH₂Br₂ is added at room temperature and the reaction mixture stirred overnight. The resulting solid is washed with water and may be purified by column chromatography.

### EXAMPLE 2

### Preparation of PTA Pentylene Dimer—Formula (III)

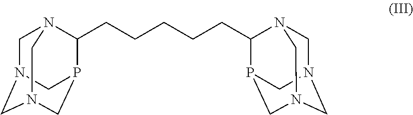

### Synthetic Scheme:

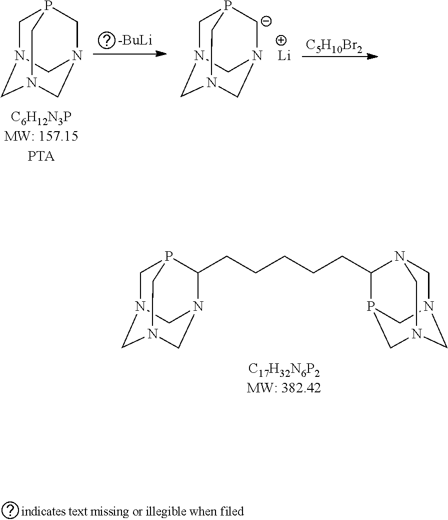

Raw materials:

| | | Water | |
|---|---|---|---|
| Material | Purity/strength | content | Origin |
| PTA | P-NMR > 99% | / | LGJ295 |
| THF | 99% | 0.04% | HONEYWELL |
| n-Bu—Li | 2.5M | / | HUALUN CO. |
| C₅H₁₀Br₂ | 99% | 0.041% | SCRC |

### Procedure and Results:

### Batch A:

A 500 ml flask sealed with nitrogen was charged with 18.6 g PTA, 270 ml THF (dried). 53 ml 2.5M n-BuLi in n-hexane was added drop wise into the mixture between −34 to −12° C. over a period of 55 minutes.

The temperature of the reaction mixture was then allowed to rise naturally to room temperature and the reaction mixture was stirred for 3 hours. It was observed to be an off-white suspension. The reaction mixture was checked by P-NMR.

Then 10.4 g C₅H₁₀Br₂ was added between −28 to −20° C. over a period of 20 mins. The reaction mixture was then stirred, for a total of 26 hours, at room temperature. It was observed to be a yellow suspension. The next day it was checked by P-NMR.

Then 14 ml H₂O was added. After separation the water phase product (observed to be a yellow viscous solid) was extracted with DCM (500 ml×5) and the organic phase was dried over Na₂SO₄, filtered, evaporated and 15 g solid was obtained.

The crude product was purified by silica gel, DCM:EA=(4:1; 3:1; 2:1; 1:1). A first sample of 2 g solid was obtained, (P-NMR-97%), and a second sample, of another 1 g solid, was obtained (P-NMR=93%).

### Batch B:

The procedure of Batch A was repeated but with the variations indicated in Table 1:

TABLE 1

| | | | n-BuLi, | | |
|---|---|---|---|---|---|
| Batch | PTA | Solvent | 2.5M | C₅H₁₀Br₂ | Temp |
| A | 18.6 g, | THF, | 53 ml, | 10.4 g, 0.38eq | RT, 26 hs |
| | 0.118 mol | 270 ml | 1.1eq | | |
| B | 12.4 g, | THF, | 37.4 ml, | 6.94 g, 0.38eq | RT, 24 hs |
| | 0.078 mol | 180 ml | 1.2eq | | |

### Results:

The products were weighed and analysed for purity with P-NMR. The results are shown in Table 2:

TABLE 2

| Batch | Solid | P-NMR purity |
|---|---|---|
| A - sample 1 | 2 g | 95% |
| A - sample 2 | <1.0 g | 93% |
| B | 1.1 g | 93% |

The product of Batch A—sample 1 was analysed by ³¹P-NMR, ¹H-NMR, ¹³C-NMR, COSY, HMBC and HSQC to confirm the product as 1,5-di{1,3,5-triaza-7-phosphatricyclo[3,3,1,1]}pentane (C₁₇H₃₂N₆P₂), in accordance with formula (III).

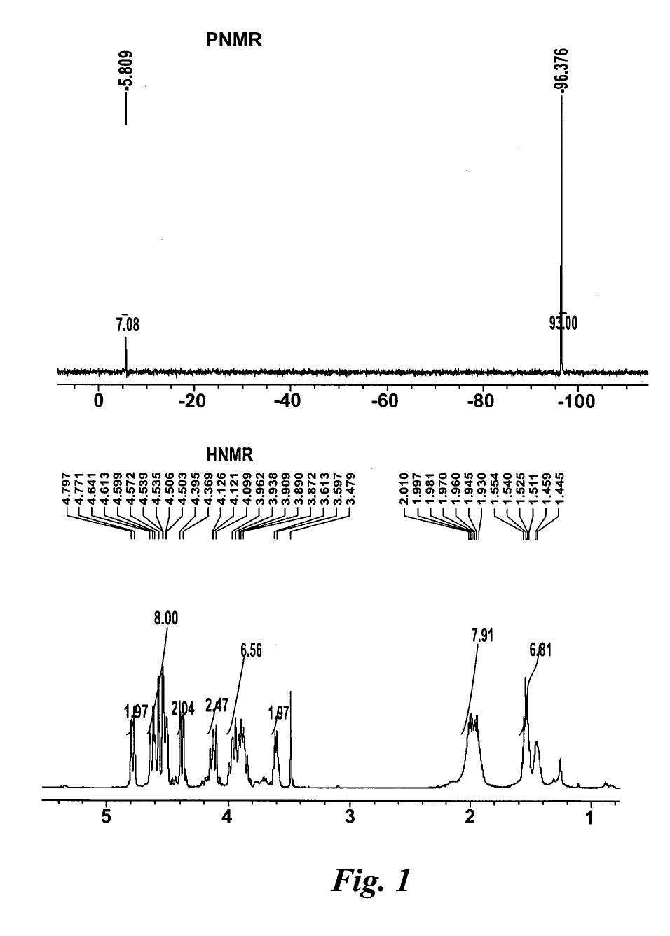

The product of Batch B was analysed by ³¹P-NMR, and ¹H-NMR to confirm the product as 1,5-di{1,3,5-triaza-7-phosphatricyclo[3,3,1,1]}pentane (C₁₇H₃₂N₆P₂), in accordance with formula (III). The NMR spectra are shown in FIG. 1.

The product of the example can be used to form metal complexes, such as PdCl₂ (C₁₇H₃₂N₆P₂) and PtCl₂ (C₁₇H₃₂N₆P₂).

1. A caged phosphine product which is a compound of formula (I): 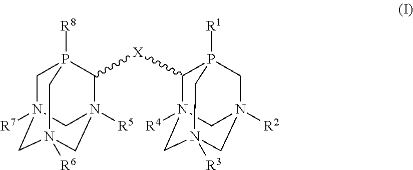
or a salt thereof;
wherein the groups R¹ and R⁸ each independently represent a substituent group selected from the group consisting of:
(a) no substituent group;
(b) an oxide substituent group ═O;
(c) a sulphide substituent group ═S;
(d) a selenide substituent group ═Se;
(e) a C1-C8 alkyl substituent group;
(f) a C6-C8 aryl substituent group or a 5 to 8 membered ring hetero aryl substituent group; and
(g) a Lewis acid substituent group;
wherein the groups R², R³, R⁴, R⁵, R⁶ and R⁷ each independently represent a substituent group selected from the group consisting of:
(1) no substituent group;
(2) a hydrogen substituent group;
(3) a C1-C8 alkyl substituent group;
(4) a C1-C8 alkoxy substituent group; and
(5) a C1-C8 acyl substituent group;
and wherein X represents a linking group that is selected from the group consisting of:
(i) a C1-C12 alkylene linking group;
(ii) an ether linking group;
(iii) a C2-C6 alkenylene linking group;
(iv) an ester linking group;
(v) a (hetero)arylene linker;
(vi) an amine linker; and
(vii) a thioether linker.

2. The product of claim 1 wherein the groups R¹ and R⁸ each independently represent a substituent group selected from the group consisting of:
(a) no substituent group;
(b) an oxide substituent group ═O;
(c) a sulphide substituent group ═S;
(d) a selenide substituent group ═Se; and
(e) a C1-C4 alkyl substituent group.

3. The product of claim 2 wherein the groups R¹ and R⁸ each represent no substituent group.

4. The product of claim 1 wherein all of R², R³, R⁴, R⁵, R⁶ and R⁷ represent no substituent group.

5. The product of claim 1 wherein four or five of R², R³, R⁴, R⁵, R⁶ and R⁷ represent no substituent group, whilst the remainder are selected from hydrogen substituent groups or C1-C8 alkyl substituent groups.

6. The product of claim 1 wherein the groups R², R³, R⁴, R⁵, R⁶ and R⁷ each independently represent a substituent group selected from the group consisting of:
(1) no substituent group;
(2) a hydrogen substituent group;
(3) a C1-C4 alkyl substituent group;
(4) a C1-C4 alkoxy substituent group; and
(5) a C1-C4 acyl substituent group.

7. The product of claim 1 wherein X represents a linking group that is a C1-C12 alkylene linking group.

8. The product of claim 1 wherein X represents a linking group that is selected from the group consisting of:
(i) a C1-C6 alkylene linking group;
(ii) an ether linking group;
(iii) a C2-C4 alkenylene linking group;
(iv) an ester linking group;
(v) a (hetero)arylene linker;
(vi) an amine linker; and
(vii) a thioether linker.

9. The product of claim 1 wherein X represents a linking group that is a C1-C6 alkylene linking group.

10. The product of claim 1 wherein the groups R², R³, R⁴, R⁵, R⁶, and R⁷ each independently represent: no substituent group or a C1-C4 alkyl substituent group, and X represents a linking group that is a C1-C12 alkylene linking group.

11. A metal complex comprising a metal M coordinated with a ligand which is a compound of formula (I): 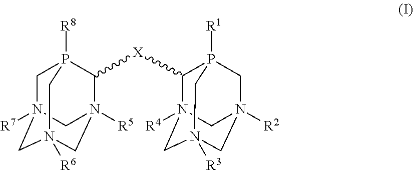
or a salt thereof;
wherein the groups R¹ and R⁸ each independently represent a substituent group selected from the group consisting of:
(a) no substituent group;
(b) an oxide substituent group ═O;
(c) a sulphide substituent group ═S;
(d) a selenide substituent group ═Se;
(e) a C1-C8 alkyl substituent group;
(f) a C6-C8 aryl substituent group or a 5 to 8 membered in hetero aryl substituent group; and
(g) a Lewis acid substituent group;
wherein the groups R², R³, R⁴, R⁵, R⁶ and R⁷ each independently represent a substituent group selected from the group consisting of:
(1) no substituent group;
(2) a hydrogen substituent group;
(3) a C1-C8 alkyl substituent group;
(4) a C1-C8 alkoxy substituent group; and
(5) a C1-C8 acyl substituent group;
and wherein X represents a linking group that is selected from the group consisting of:
(i) a C1-C12 alkylene linking group;
(ii) an ether linking group;
(iii) a C2-C6 alkenylene linking group;
(iv) an ester linking group;
(v) a (hetero)arylene linker;
(vi) an amine linker; and
(vii) a thioether linker.

12. The metal complex of claim 11 wherein the metal M is a transition metal.

13. The metal complex of claim 12, wherein the metal M is selected from the group consisting of: rhodium, ruthenium, rhenium, iridium, cobalt, nickel, platinum and palladium.

14. A method of catalysing a reaction, the method comprising the steps of:
providing a caged phosphine product which is a compound of formula (1); 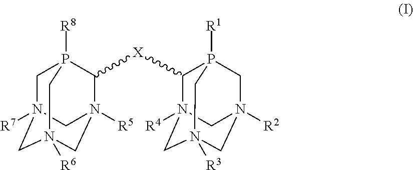
or a salt thereof;
wherein the groups R¹ and R⁸ each independently represent a substituent group selected from the group consisting of:
(a) no substituent group;
(b) an oxide substituent group ═O;
(c) a sulphide substituent group ═S;
(d) a selenide substituent group ═Se;
(e) a C1-C8 alkyl substituent group;
(f) a C6-C8 alkyl substituent group or a 5 to 8 membered in hetero aryl substituent group; and
(g) a Lewis acid substituent group;
wherein the groups R², R³, R⁴, R⁵, R⁶ and R⁷ each independently represent a substituent group selected from the group consisting of:
(1) no substituent group;
(2) a hydrogen substituent group;
(3) a C1-C8 alkyl substituent group;
(4) a C1-C8 alkoxy substituent group; and
(5) a C1-C8 acyl substituent group;
and wherein X represents a linking group that is selected from the group consisting of:
(i) a C1-C12 alkylene linking group;
(ii) an ether linking group;
(iii) a C2-C6 alkenylene linking group;
(iv) an ester linking group;
(v) (hetero)arylene linker;
(vi) an amine linker; and
(vii) a thioether linker;
and
using the complex as a catalyst for the reaction.

15. The method of claim 14, wherein the catalyst is used to catalyse an organic reaction.
